(19) United States
(12) Patent Application Publication
(10) Pub. No.: US 2009/0149662 A1
(43) Pub. Date: Jun. 11, 2009

(54) PROCESSES FOR PREPARING ZAFIRLUKAST

(76) Inventors: Raghupathi Reddy Anumula, Hyderabad (IN); Goverdhan Gilla, Hyderabad (IN); Sampath Alla, Karimnagar (IN); Sreenivasulu Kurella, Nalgonda (IN); Janardana Sarma Ramachandra Kopparapu, Secunderabad (IN); Rama Krishna Venkata Medisetti, Visakhapatnam (IN); Srinivasula Reddy Maddula, Guntur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/328,374

(22) Filed: Dec. 4, 2008

Related U.S. Application Data
(60) Provisional application No. 61/082,722, filed on Jul. 22, 2008.

(30) Foreign Application Priority Data
Dec. 5, 2007 (IN) 2899/CHE/2007

(51) Int. Cl. C07D 209/18; C07D 403/06; C07D 403/12; G01N 21/00

(52) U.S. Cl. 548/455; 548/510; 436/164

(57) ABSTRACT

An improved process for the preparation of substantially pure zafirlukast and pharmaceutical compositions thereof.

The present application relates to an improved process for the preparation of substantially pure zafirlukast and pharmaceutical compositions thereof.

Zafirlukast is 4-(5-cyclopentyloxy-carbonylamino-1-methyl-indol-3-ylmethyl)-3-methoxy-N-O-tolylsulfonylbenzamide and represented by the structural Formula I.

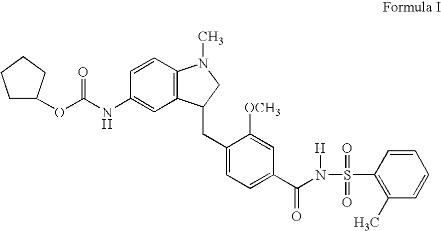

Zafirlukast is an oral leukotriene receptor antagonist useful for the treatment of asthma.

U.S. Pat. No. 4,859,692 discloses zafirlukast, a process for its preparation, a pharmaceutical composition containing zafirlukast, and its use as a leukotriene antagonist.

U.S. Pat. No. 5,319,097 discloses polymorphic Form A (amorphous), Form B, and Form X of zafirlukast, and processes for their preparation.

### SUMMARY

The present invention includes processes for the preparation of methyl 3-methoxy-4-(1-methyl-5-nitro-1H-indol-3-ylmethyl)benzoate of Formula VI, which is a key intermediate in the preparation of zafirlukast. The processes comprise at least one of the steps of:
(1) brominating methyl 3-methoxy-4-methylbenzoate of Formula IX using a mixture of hydrogen bromide and a peroxide in the presence of a suitable organic solvent to afford methyl 4-bromomethyl-3-methoxybenzoate of Formula VIII; and

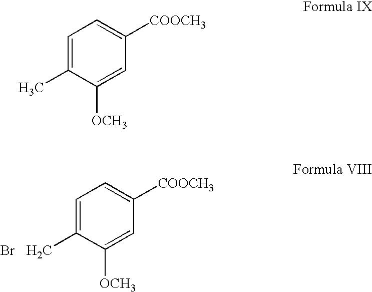

(2) condensing methyl 4-bromomethyl-3-methoxybenzoate of Formula VIII with 1-methyl-5-nitro-indole of Formula VII in the presence of a suitable catalyst to afford methyl 3-methoxy-4-((1-methyl-5-nitro-1H-indol-3-yl)methyl)-benzoate of Formula VI.

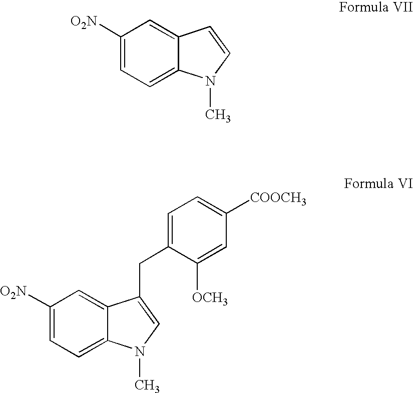

The present invention includes processes for the purification of methyl 4-((5-amino-1-methyl-1H-indol-3-yl)methyl)-3-methoxybenzoate of Formula V, which processes comprise recrystallization from an organic solvent.

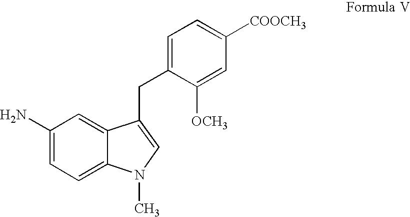

The present invention includes processes for the purification of zafirlukast, which processes include treating crude zafirlukast with an adsorbent to afford zafirlukast substantially free of process related impurities.

The present invention includes the zafirlukast-related, structurally isomeric compounds of Formulas XI through XVIII, as well as their use as references/standard markers in the HPLC analysis of zafirlukast and its salts.

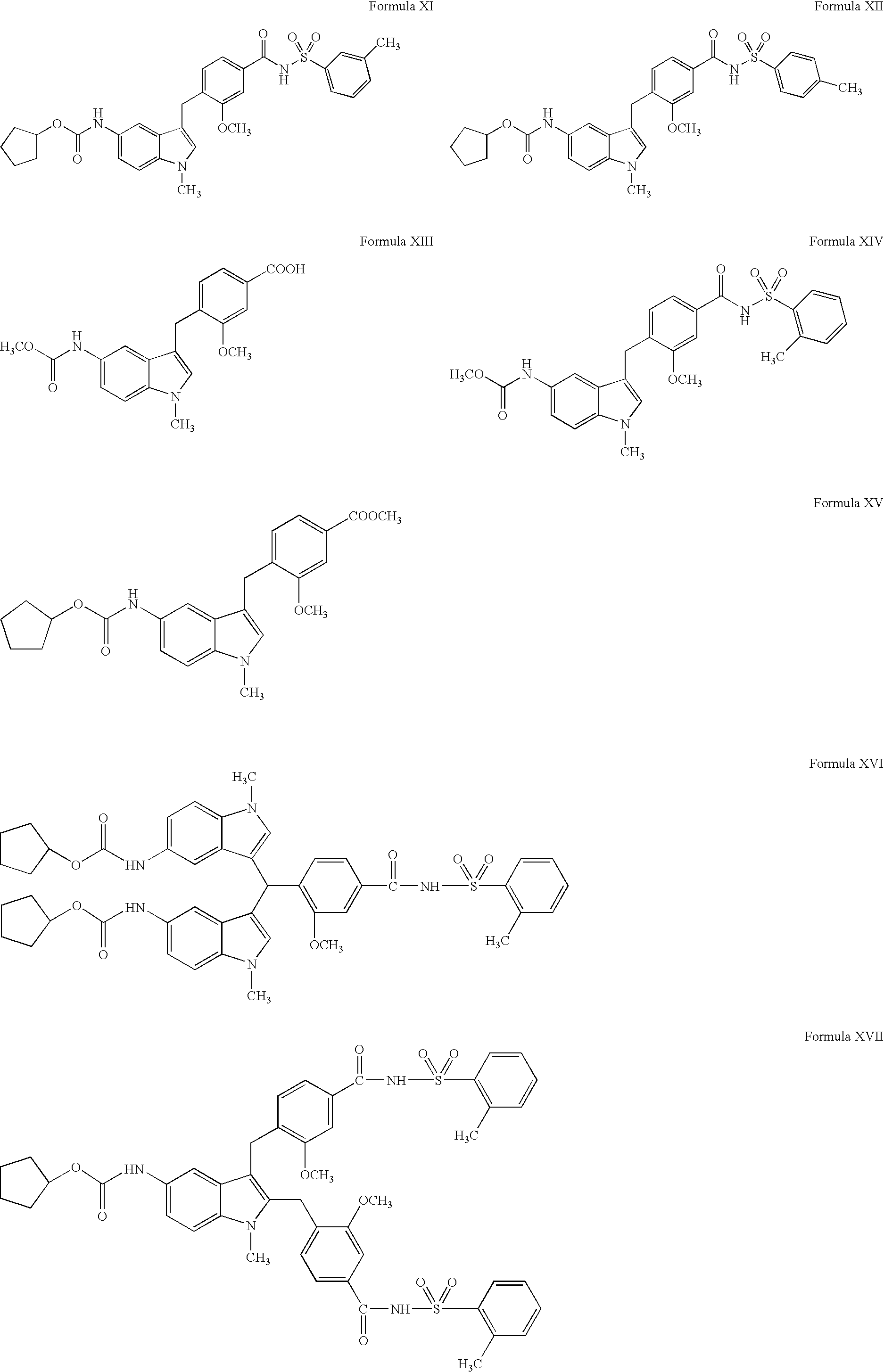

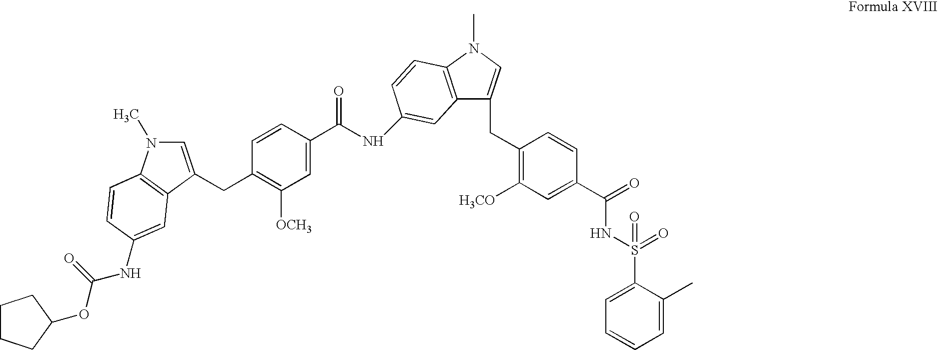

The present invention includes processes for the preparation of crystalline Form X of zafirlukast, which processes comprise at least one of the steps of:
(1) providing a solution of zafirlukast in ethyl acetate;
(2) cooling the solution to precipitate a solid; and
(3) isolating the solid which is the crystalline Form X of zafirlukast.

The present invention includes processes for the preparation of amorphous zafirlukast, which processes comprise at least one of the steps of:
(1) providing a solution of zafirlukast in a volatile solvent; and
(2) removing the solvent by spray drying or rotatory evaporation to afford the amorphous form of zafirlukast.

### BRIEF DESCRIPTION OF THE DRAWINGS

FIG. 1 is a schematic representation of a process according to the present invention for the preparation of the compound of Formula V.

FIG. 2 is a schematic representation of a process according to the present invention for the preparation of the compound of Formula I.

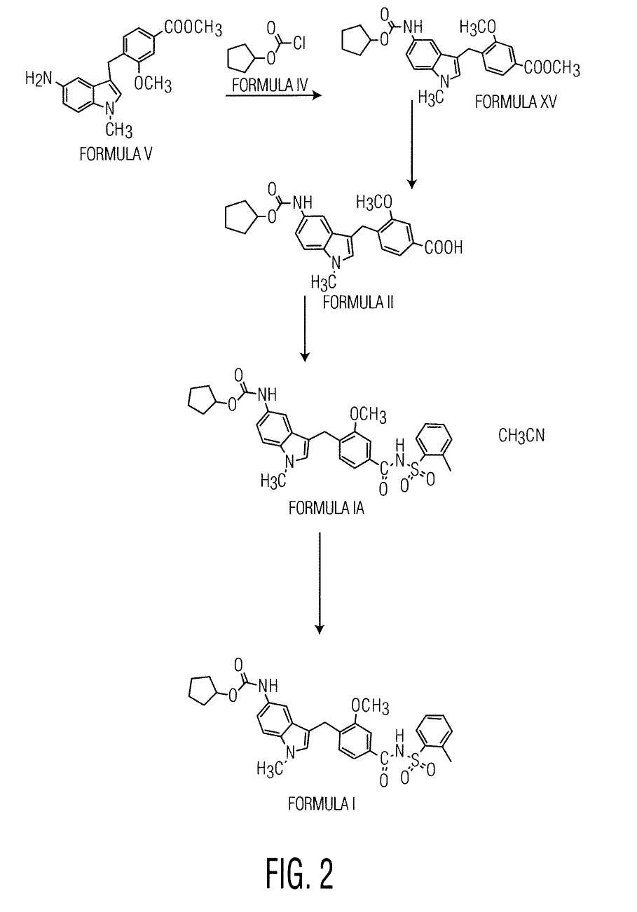

FIG. 3 is an X-ray powder diffraction (XPRD) pattern of zafirlukast acetonitrile solvate prepared as per Example 9.

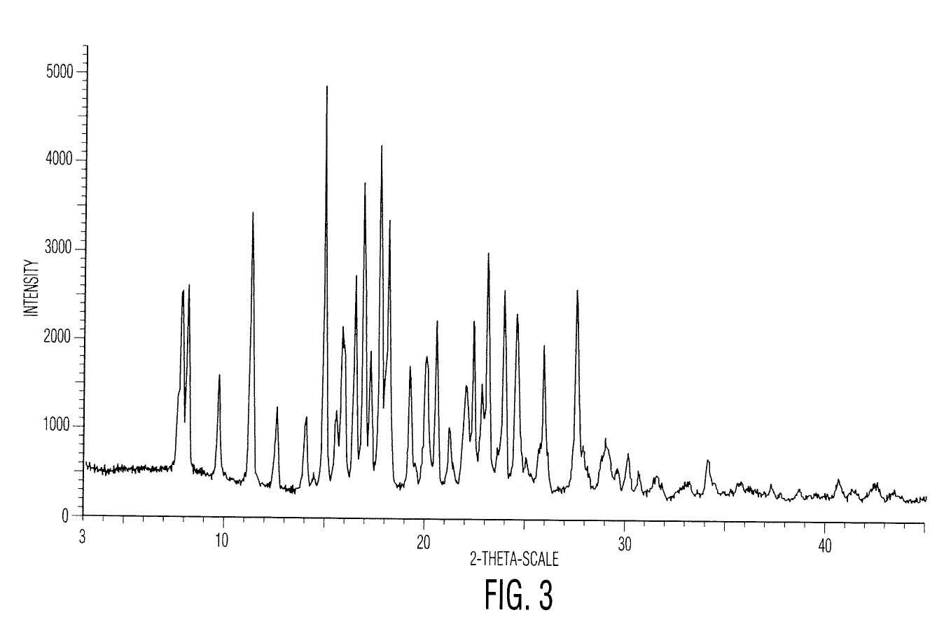

FIG. 4 is a DSC pattern of zafirlukast acetonitrile solvate prepared as per Example 9.

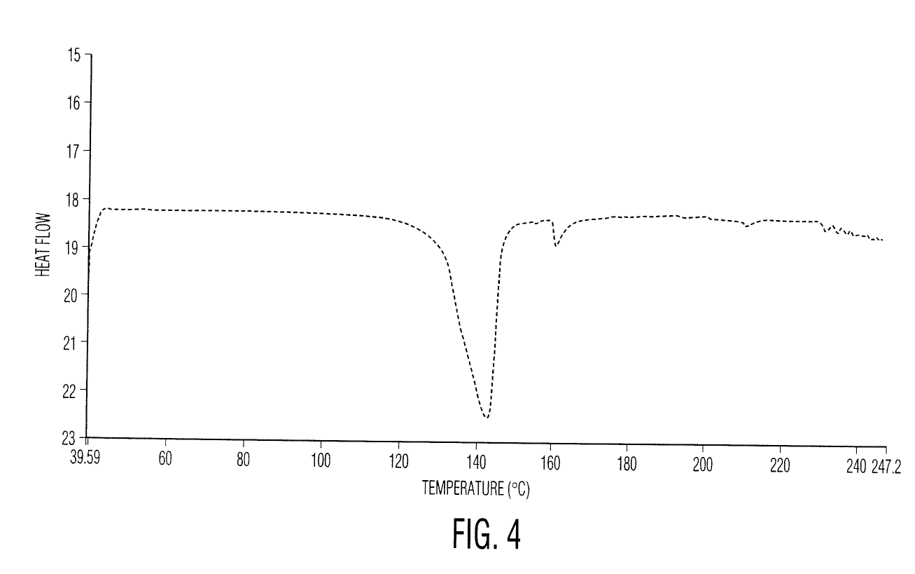

FIG. 5 is a TGA pattern of zafirlukast acetonitrile solvate prepared as per Example 9.

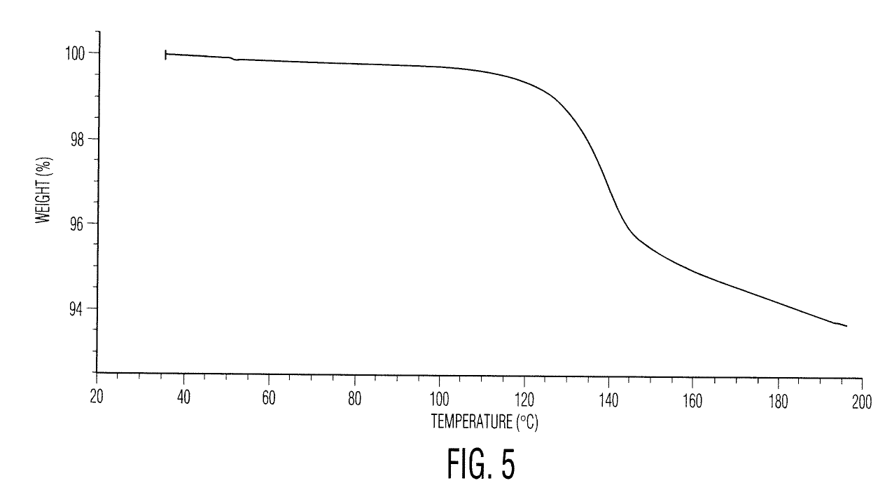

### DETAILED DESCRIPTION

The present invention includes processes for the preparation of methyl 3-methoxy-4-(1-methyl-5-nitro-1H-indol-3-ylmethyl)benzoate of Formula VI, which is a key intermediate in the preparation of zafirlukast. The processes comprise at least one of the steps of:
(1) brominating methyl 3-methoxy-4-methylbenzoate of Formula IX using a mixture of hydrogen bromide and a peroxide in the presence of a suitable organic solvent to afford methyl 3-methoxy-4-bromomethylbenzoate of Formula VIII; and

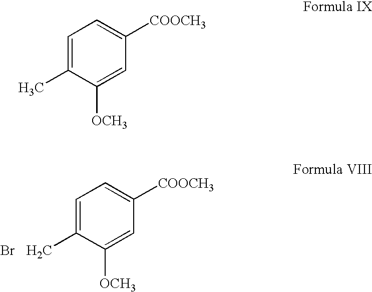

(2) condensing methyl 3-methoxy-4-bromomethylbenzoate of Formula VIII with 1-methyl-5-nitro-1H-indole of Formula VII in the presence of a suitable catalyst to afford methyl 3-methoxy-4-(1-methyl-5-nitro-1H-indol-3-yl)methylbenzoate of Formula VI.

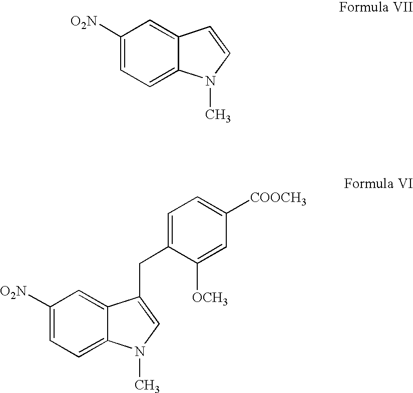

Step (1) involves brominating methyl 3-methoxy-4-methylbenzoate of Formula IX using a mixture of hydrogen bromide and a peroxide in the presence of a suitable organic solvent to afford methyl 3-methoxy-4-bromomethylbenzoate of Formula VIII

The solution of methyl 3-methoxy-4-methylbenzoate may be obtained by dissolving methyl 3-methoxy-4-methylbenzoate in a solvent, or such a solution may be used directly from a reaction in which methyl 3-methoxy-4-methylbenzoate is formed.

Suitable solvents that may be used for dissolution of methyl 3-methoxy-4-methylbenzoate include and are not limited to, alcohols, such as, for example, methanol, ethanol, isopropanol, and n-butanol; ketones, such as, for example, acetone, ethyl methyl ketone, and methyl isobutyl ketone; esters, such as, for example, ethyl acetate, n-propyl acetate, n-butyl acetate, and t-butyl acetate; nitriles, such as, for example, acetonitrile, and propionitrile; halogenated hydrocarbons, such as, for example, dichloromethane, ethylene dichloride, and chloroform; hydrocarbons, such as, for example, benzene, toluene, hexane, and cyclohexane; aprotic solvents, such as, for example, N,N-dimethylformamide (DMF), dimethylsulfoxide (DMSO), and N,N-dimethylacetamide (DMA); and mixtures thereof in various proportions.

The brominating agent that may be used in this reaction is a combination of hydrogen bromide and a peroxide. Non-limiting examples of suitable peroxides include hydrogen peroxide and benzyl peroxide. The ratio of hydrogen bromide to hydrogen peroxide may range from about 1 to about 5 times or between 1 to about 3. The molar ratio of hydrogen bromide (45%) to methyl 3-methoxy-4-methylbenzoate may range from about 1:1 to 5:1, or between 1.1:1 to about 1.5:1.

The duration of the reaction can be from about 30 minutes to about 4 hours, or for about 60 minutes. The temperature at which the peroxide is added in Step (1) may range from about 0° C. to about 50° C., or from about 25° C. to about 30° C. If the reaction is conducted at a higher temperature and/or for a longer duration, formation of side products and process-related impurities might increase.

Step (2) involves condensing methyl 3-methoxy-4-bromomethylbenzoate of Formula VIII with 1-methyl-5-nitro-1H-indole of Formula VII to afford methyl 3-methoxy-4-(1-methyl-5-nitro-1H-indol-3-yl)methylbenzoate of Formula VI

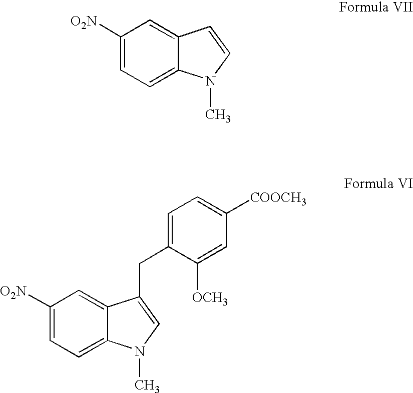

The compound of Formula VIII may be condensed with the compound of Formula VII in the presence of a catalyst in an organic solvent. Examples of catalysts include and are not limited to copper oxide, zinc oxide, magnesium oxide, and calcium oxide.

Suitable organic solvents that may be used in Step (2) include and are not limited to halogenated solvents, such as, for example, dichloromethane, dichloroethane, and chloroform; ethers, such as, for example, methyl tert-butyl ether, tetrahydrofuran, and 1,4-dioxane; hydrocarbons, such as, for example, toluene, xylene, n-hexane, n-heptane, and cyclohexane; and mixtures thereof.

The temperature for conducting the reaction may range from about 50° C. to about 120° C. and the duration of the reaction may range from about 5 hours to about 50 hours, or from about 24 hours to about 30 hours.

The present invention includes processes for the purification of methyl 3-methoxy-4-(1-methyl-5-amino-1H-indol-3-yl)methylbenzoate of Formula V, which processes comprise recrystallization from an organic solvent.

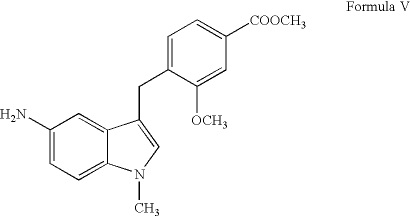

The compound of Formula V may either be in the form of a free base or an acid addition salt.

The acid addition salt may be recrystallized by using an organic solvent. Suitable organic solvents include and are not limited to: alcohols, such as, for example, methanol, ethanol, isopropanol, and n-butanol; ketones, such as, for example, acetone, ethyl methyl ketone, and methyl isobutyl ketone; esters, such as, for example, ethyl acetate, n-propyl acetate, n-butyl acetate, and t-butyl acetate; nitrites, such as, for example, acetonitrile, and propionitrile; halogenated hydrocarbons, such as, for example, dichloromethane, ethylene dichloride, and chloroform; hydrocarbons, such as, for example, benzene, toluene, hexane, and cyclohexane; aprotic solvents, such as, for example, DMF, DMSO, and DMA; and mixtures thereof in various proportions. For example, the organic solvent for the crystallization of the compound of Formula V and its salts is ethyl acetate.

The compound of Formula V that is purified according to the present process is substantially free from process-related impurities. The pure methyl 3-methoxy-4-(1-methyl-5-amino-1H-indol-3-yl)methylbenzoate of Formula V obtained by the above process has a purity equal or greater than 99% by HPLC.

The present invention includes processes for the purification of zafirlukast, which processes include treating crude zafirlukast with an adsorbent to afford zafirlukast substantially free of process related impurities.

Non-limiting examples of suitable adsorbent materials include silica gel, aluminum oxide, and synthetic resin. The ratio of adsorbent to crude zafirlukast may range from about 1 to 5 times or about 2 times.

The adsorbent may be added to a solution of crude zafirlukast. The solution may be obtained directly by dissolving crude zafirlukast in a suitable solvent or the solution may be a reaction mixture containing zafirlukast that is obtained during preparation thereof. Suitable solvents that may be used for dissolution of zafirlukast include and are not limited to alcohols, such as, for example, methanol, ethanol, isopropanol, and n-butanol; ketones, such as, for example, acetone, ethyl methyl ketone, and methyl isobutyl ketone; esters, such as, for example, ethyl acetate, n-propyl acetate, n-butyl acetate, and t-butyl acetate; nitriles, such as, for example, acetonitrile, and propionitrile; halogenated hydrocarbons, such as, for example, dichloromethane, ethylene dichloride, and chloroform hydrocarbons, such as, for example, benzene, toluene, hexane, and cyclohexane; aprotic solvents, such as, for example, DMF, DMSO, and DMA; and mixtures thereof in various proportions. The amount of solvent that may be used to dissolve the crude zafirlukast may range from about 10 times to about 15 times to the weight of crude zafirlukast. The temperature for dissolution can range from about 25° C. to about 100° C., or the reflux temperature of the solvent used.

The obtained reaction solution may be filtered, the filter cake may be washed with a solvent, and the solvent may be distilled off from the filtrate to get pure zafirlukast.

The zafirlukast thus obtained may be further purified by recrystallization using a nitrile solvent. Suitable nitrile solvents that may be used include and are not limited to acetonitrile and propionitrile. The obtained zafirlukast may be in a solvated form.

The pure zafirlukast of Formula I obtained by the above process may have a purity equal to or greater than 99% or 99.9% or by HPLC.

For the HPLC analysis, an octadecyl silyl silica gel column packed with 5 μm particles or equivalent is used. Other parameters are listed in Table 1.

TABLE 1

| Flow rate | 0.8 mL/min | | | |
|---|---|---|---|---|
| Wavelength | 220 nm | | | |
| Injection load | 20 μl | | | |
| Temperature | 27° C. | | | |
| Run Time | 85 min | | | |
| Diluent | Acetonitrile:Water (8:2) | | | |
| Buffer | 2.72 g of KH₂PO₄ and 1 g of 1-decane sulphonic | | | |
| preparation | acid sodium salt in 1000 mL of purified water pH | | | |
| | adjusted to 4 with dilute phosphoric acid (1 in 10 mL) | | | |
| Mobile phase | Eluent A; degassed buffer | | | |
| preparation | Eluent B: degassed mixture of acetonitrile, methanol, | | | |
| | and water in a ratio of 85:10:5 | | | |
| | | | | Mode of |
| | Time | Eluent A | Eluent B | gradient |
| Gradient program | 00-35 | 60-40 | 40-60 | Linear |
| | | | | gradient |
| | 45-55 | 26 | 74 | Isocratic |
| | 60-65 | 14-7 | 86-93 | Linear |
| | | | | gradient |
| | 75-85 | 60 | 40 | Switch to |
| | | | | initial elution |
| | | | | composition |

A number of impurities were identified and each is contemplated for the purpose of use as as references/standard markers in the HPLC analysis of zafirlukast and its salts.

A zafirlukast-related impurity is {3-[2-methoxy-4-(toluene-3-sulfonylaminocarbonyl)-benzyl]-1-methyl-1H-indol-5-yl}-carbamic acid cyclopentyl ester of Formula XI with a relative retention time (RRT) of about 1.03 minutes in the HPLC method set forth above:

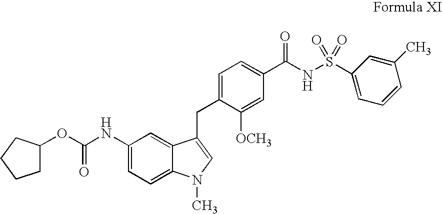

A zafirlukast related impurity is {3-[2-methoxy-4-(toluene-4-sulfonylaminocarbonyl)-benzyl]-1-methyl-1H-indol-5-yl}-carbamic acid cyclopentyl ester of Formula XII with a relative retention time of about 1.06 minutes in the HPLC method set forth above.

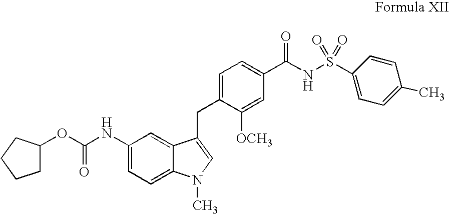

A zafirlukast-related impurity is {3-[2-methoxy-4-carboxy-benzyl]-1-methyl-1H-indol-5-yl}-carbamic acid methyl ester of Formula XIII with a relative retention time of about 0.71 minutes in the HPLC method set forth above.

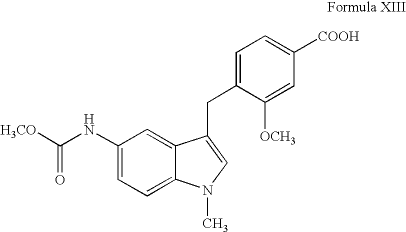

A zafirlukast-related impurity is {3-[2-methoxy-4-(toluene-2-sulfonylaminocarbonyl)-benzyl]-1-methyl-1H-indol-5-yl}-carbamic acid methyl ester of Formula XIV with a relative retention time of about 0.56 minutes in the HPLC method set forth above.

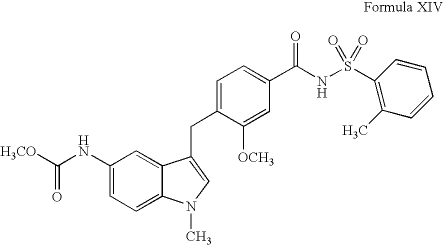

A zafirlukast-related impurity is {3-[2-methoxy-4-methoxycarbonyl-benzyl]-1-methyl-1H-indol-5-yl}-carbamic acid cyclopentyl ester of Formula XV with a relative retention time of about 1.21 minutes in the HPLC method set forth above.

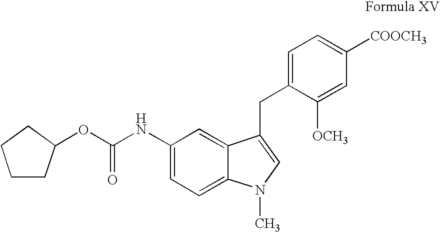

A zafirlukast-related impurity is (4-[bis-(5-cyclopentyloxycarbonyl-amino-1-methyl-1H-indol-3-yl)-methyl]-3-methoxy-benzoyl)-(2-methyl-benzene)sulfonamide of Formula XVI with a relative retention of time about 1.19 minutes in the HPLC method set forth above.

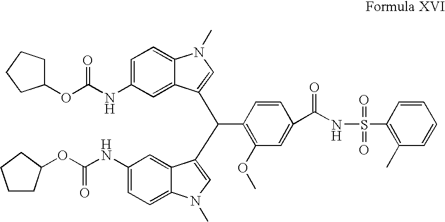

A zafirlukast-related impurity is {2,3-di[2-methoxy-4-(toluene-2-sulfonylaminocarbonyl)-benzyl]-1-methyl-1H-indol-5-yl}-carbamic acid cyclopentyl ester of Formula XVII with a relative retention time of about 1.18 minutes in the HPLC method set forth above.

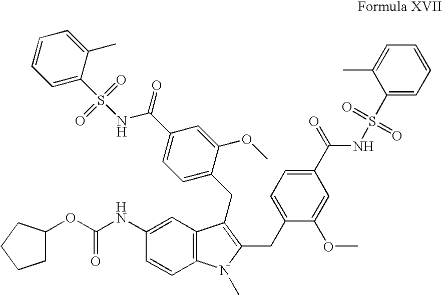

A zafirlukast-related impurity is [3-(2-methoxy-4-{3-[2-methoxy-4-(toluene-2-sulfonylaminocarbonyl)-benzyl]-1-methyl-1H-indol-5-ylcarbamoyl}-benzyl)-1-methyl-1H-indol-5-yl]-carbamic acid cyclopentyl ester of Formula XVIII with a relative retention time of about 1.22 minutes in the HPLC method set forth above.

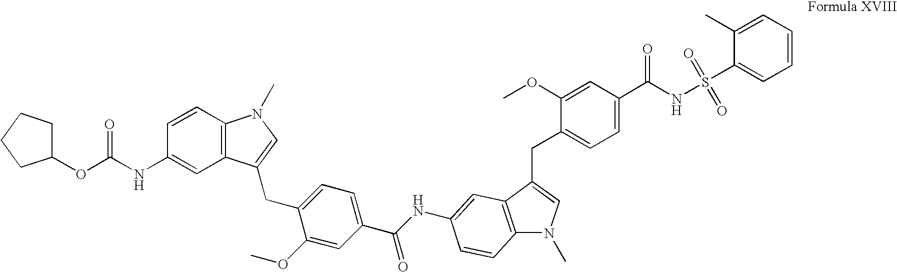

The above-described zafirlukast-related impurities are useful as reference standards/markers to analyze zafirlukast or its related intermediates. For example, a known quantity of a reference standard/marker may be injected into a detector to quantify the detector response to that reference standard/marker. In addition, a reference standard/marker may be used for qualitative analysis by matching the RRT (relative retention time) of a reference standard with a known chemical structure with the RRTs of impurities present in the analyte. Moreover, the detection or quantification of reference standards/markers serves to establish the level of purity of the API or intermediates thereof.

The present invention includes processes for the preparation of crystalline Form X of zafirlukast. Form X is described in U.S. Pat. No. 5,319,097, which is incorporated herein by reference in its entirety. The processes of the present invention involve recrystallization of zafirlukast from an organic solvent.

A solution of zafirlukast and an organic solvent may be obtained by dissolving zafirlukast in the organic solvent, or such a solution may be used directly from a reaction in which zafirlukast is formed. The starting zafirlukast may be a crystalline, amorphous, or solvated form, or a mixture of crystalline, amorphous, or solvated form. The temperature for dissolution may range from about 25° C. to about 100° C. The time period may be as long as required for complete dissolution.

Once the solution is formed, if desired, typical intermediate steps may be undertaken to remove undissolved particles, treat the solution with activated carbon, etc. The clear solution is then handled to reduce solubility of dissolved zafirlukast, e.g., by cooling. The time period for such handling can be as long as required for the complete precipitation of zafirlukast.

Recovery of solid may be carried out by conventional techniques such as filtration, decantation, and centrifugation in the presence or absence of inert atmosphere.

The wet cake may be further dried. Drying may be suitably carried out in a tray dryer, vacuum oven, air oven, fluidized bed drier, spin flash dryer, flash dryer, and the like. The drying may be carried out at temperatures of about 35° C. to about 90° C. with or without vacuum. The drying can be carried out for any desired time until the required product purity is achieved.

The present invention includes processes for the preparation of amorphous zafirlukast, which processes comprise:
(1) providing a solution of zafirlukast in a volatile solvent; and
(2) removing the solvent by spray drier or rotary evaporation to afford the amorphous form of zafirlukast.

Suitable solvents that may be used for dissolution of zafirlukast include and are not limited to: ketonic solvents, such as, for example, C₂-C₅ ketones; halogenated solvents, such as, for example, dichloromethane and chloroform; and mixtures thereof.

The temperature for dissolution may range from about 25° C. to about 100° C. or reflux temperature of the solvents used.

Recovery of solid may be carried out by conventional techniques, such as, for example, spray drying or rotary evaporation in the presence or absence of an inert atmosphere.

The wet cake may be further dried. Drying may be carried out in a tray dryer, vacuum oven, air oven, fluidized bed drier, spin flash dryer, flash dryer, and the like. The drying may be carried out at temperatures of from about 35° C. to about 90° C. with or without vacuum. The drying may be carried out for any desired time until the required product purity is achieved.

The present invention includes zafirlukast of Formula I having a mean particle size of less than or equal to about 300 μm.

The D₁₀, and D₉₀ values are useful ways for indicating a particle size distribution. D₉₀ refers to at least 90 volume percent of the particles having a size smaller than the said value. Likewise D₁₀ refers to 10 volume percent of the particles having a size smaller than the said value. D₅₀ refers to at least 50 volume percent of the particles having a size smaller than the said value. Methods for determining D₁₀, D₅₀ and D₉₀ include laser diffraction using Malvern equipment.

Zafirlukast according to the present invention has a D₁₀ of less than about 10 μm, or less than about 5 μm; a D₅₀ of less than about 50 μm, or less than about 30 μm, or less than about 10 μm.; and a D₉₀ of less than about 200 μm, or less than about 125 μm. There is no specific lower limit for any of the D values.

The present invention includes pharmaceutical compositions and method of making the same, the pharmaceutical compositions comprising: zafirlukast that is substantially free of impurities made by the processes of the present invention, and at least one pharmaceutically acceptable excipient.

The drug substance can be formulated as a solid composition for oral administration. For example, the drug substance may be formulated as a tablet, pill, powder, or granule. The drug substance can be formulated as a liquid composition for oral or parenteral administration. For example, the drug substance may be formulated as a solution, suspension, syrup, elixir, or emulsion. If required, sterilization of the liquid composition may be carried out in several ways, such as, for example, using a bacteriological filter, by incorporating sterilizing agents in the composition, by irradiation or by heating.

Pharmaceutically acceptable excipients that are of useful in the present invention include and are not limited to: diluents, such as, for example, starch, pregelatinized starch, lactose, powdered cellulose, microcrystalline cellulose, dicalcium phosphate, tricalcium phosphate, mannitol, sorbitol, sugar and the like; binders, such as, for example, acacia, guar gum, tragacanth, gelatin, polyvinyl pyrrolidone, hydroxypropyl cellulose, hydroxypropyl methyl cellulose, pregelatinized starch and the like; disintegrants, such as, for example, starch, sodium starch glycolate, pregelatinized starch, crospovidone, croscarmellose sodium, colloidal silicon dioxide and the like; lubricants, such as, for example, stearic acid, magnesium stearate, zinc stearate and the like; glidants, such as, for example, colloidal silicon dioxide and the like; solubility or wetting enhancers, such as, for example, anionic or cationic or neutral surfactants, complex forming agents, such as, for example, various grades of cyclodextrins, resins; release rate controlling agents, such as, for example, hydroxypropyl cellulose, hydroxymethyl cellulose, hydroxypropyl methyl cellulose, ethyl cellulose, methyl cellulose, various grades of methyl methacrylates, waxes and the like. Other pharmaceutically acceptable excipients that are of use include but not limited to film formers, plasticizers, colorants, flavoring agents, sweeteners, viscosity enhancers, preservatives, antioxidants and the like.

The process of the present invention is simple, improved, eco-friendly, cost-effective, commercially viable, robust and reproducible on an industrial scale.

The examples are to aid in understanding the invention and are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety

### EXAMPLES

### Example 1

### Preparation of methyl 3-methoxy-4-methylbenzoate (Formula IX)

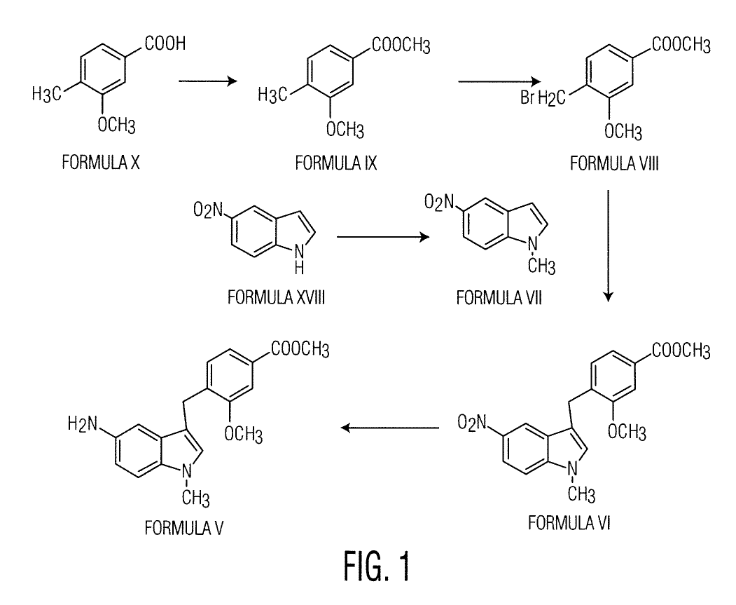

200 g of 3-methoxy-4-methyl benzoic acid of Formula X (see FIG. 1) and methanol are charged into a round neck round bottom flask and stirred for about 10 minutes. Thionyl chloride (100 mL) is added. The solution is heated to about 65° C., maintained for about 2 hours, and cooled to about 30° C. The resultant reaction solution is charged into water (1000 mL) at about 10° C. and stirred for solid separation. The solid is separated and washed with a solution of water and sodium bicarbonate. The solid is suction dried for 10 minutes to afford the title compound.

### Example 2

### Preparation of methyl 3-methoxy-4-bromomethylbenzoate (Formula VIII)

Methyl 3-methoxy-4-methylbenzoate (50 g), chloroform (500 mL), and hydrogen bromide (54.9 g) are charged into a round neck round bottom flask upon which is placed a black cover having an opening in which 200 watt light is arranged for a photochemical reaction. 30% hydrogen peroxide (94.18 g) added drop-wise over about 30 minutes followed by the drop-wise addition of sulphuric acid (81.3 mL) over about 20 minutes. The resultant reaction mass is heated to about 55° C. and stirred for about 2 hours. The reaction mass is cooled to 25° C. and a 40% sodium bicarbonate solution is added slowly by dropper and then stirred for about 15 minutes. The aqueous and organic layers are separated. The aqueous layer is washed with chloroform (2×100 mL). The obtained organic layer is combined and washed with water (2×100 mL) and re-separated. The organic layer is distilled off completely under vacuum to afford the crude. The obtained crude is cooled to 5° C. and cyclohexane is added and stirred for 2 hours for solid separation. The solid is filtered and washed with cyclohexane. The obtained solid is dried under vacuum for about 4 hours at 50° C. to afford the title compound.

### Example 3

### Alternate process for the preparation of methyl 3-methoxy-4-bromomethylbenzoate (Formula VIII)

Methyl 3-methoxy-4-methylbenzoate (7 kg) and methanol (10.5 L) are charged into a round bottom flask and stirred for about 10 minutes. Thionyl chloride (3.5 L) is added to the solution. The solution is heated to about 65° C., maintained for about 2 hours, and cooled to about 30° C. The resultant reaction solution is charged into water (35 L) at about 10° C. and stirred for solid separation. The solid is separated and washed with a solution of water (14 L) and sodium bicarbonate (14.5 L). The solid is suction dried for 10 minutes. The obtained wet compound (7.3 kg) and cyclohexane (42 L) are charged is a round bottom flask and stirred for about 30 minutes at about 30° C. followed by separation of the organic and aqueous layers. To the obtained organic layer, azo-bis-iso-butyronitrile (AIBN) (0.42 kg) and dibromodimethyl hydantoin (5.6 kg) are charged and stirred for about 30 minutes at 25° C. The resultant reaction solution is heated to about 80° C. and stirred for about 4 hours. The reaction mass is cooled to 50° C. and AIBN (0.014 kg) and dibromodimethyl hydantoin (1.4 kg) are again charged and heated to about 80° C. and stirred for about 2 hours up to completion of the reaction. The reaction mass is cooled to 60° C. and water (28 L) is charged and stirred for about 20 minutes at about 60° C. The organic and aqueous layers are separated. The aqueous layer is washed with cyclohexane (7 L). The organic layer is combined and washed with sodium bicarbonate solution (14 L) and water (14 L). The obtained clear solution is cooled to 10° C. and stirred for about 45 minutes for solid separation. The solid is filtered and washed with cyclohexane (7 L). The solid is suction dried for about 60 min. The obtained wet solid is dried to afford the 6.7 kg of the title compound.

HPLC purity: 95.44%

### Example 4

### Preparation of 1-methyl-5-nitro-1H-indole (Formula VII)

Dimethyl formamide (19.6 L) and sodium hydroxide (2.55 kg) are charged and stirred for about 30 minutes. 4.9 kg of 5-nitroindole of Formula XVIII (see FIG. 1) is added and stirred for about 30 minutes at about 30° C. Dimethyl sulfate (4.9 kg) is added and stirred for about 30 minutes at about 30° C. The resultant reaction mass is stirred for about 90 minutes at about 35° C. up to completion of the reaction. Water (49 L) is charged at about 30° C. and stirred for about 90 minutes at about 30° C. The separated solid is filtered and washed with water (24.5 L). The solid is dried under vacuum at 60° C. for about 7 hours to afford the of the title compound.

HPLC purity: 99.89%

### Example 5

### Preparation of methyl 3-methoxy-4-((1-methyl-5-nitro-1H-indol-3-yl)methyl)-benzoate (Formula VI)

1-methyl-5-nitro-1H-indole (2.5 kg), methyl 3-methoxy-4-bromomethylbenzoate (4.78 kg), and 1,4 dioxane (17.5 L) are charged and stirred for about 10 minutes. Cuprous oxide (6.1 kg) is added. The reaction solution is heated to about 100° C. and stirred for about 30 hours under nitrogen atmosphere up to completion of the reaction. The resultant reaction solution is cooled to 30° C. and passed through diatomaceous earth, which is washed with 1,4 dioxane (5 L). The obtained clear solution is distilled off completely under vacuum below 50° C. to afford the crude. To the resultant crude, ethyl acetate (2.5 L) is charged and stirred to about 50° C. for about 20 minutes followed by the addition of methanol (22.5 L). The resultant reaction solution is heated to about 50° C. and stirred for about 4 hours then cooled to about 25° C. and stirred for about 4 hours for solid separation. The solid is filtered, washed with methanol (2.5 L), and suction dried. The solid is dried under vacuum at 50° C. to afford the title compound.

### Example 6

### Preparation of methyl 3-methoxy-4-(1-methyl-5-amino-1H-indol-3-yl)methylbenzoate (Formula V)

Methyl 3-methoxy-4-((1-methyl-5-nitro-1H-indol-3-yl)methyl)-benzoate (2.9 kg), ethyl acetate (17 L), and RANEY nickel (3.0 kg) are charge into an autoclave and 6 kg hydrogen pressure is applied. The reaction mass is heated to about 35° C. and maintained for about 6 hours at 6 kg hydrogen pressure. After completion of the reaction, the raney nickel is filtered and washed with ethyl acetate (7 L). The resultant filtrates are charged and the pH of the reaction solution is adjusted to 1.5 by addition of concentrated hydrochloric acid (0.9 L). The resultant reaction mass is stirred at about 25° C. for about four hours for solid separation. The solid is filtered, washed with ethyl acetate (6 L), and suction dried. The obtained wet solid is charged into a reactor containing water (29 L) and stirred for about 10 minutes. The pH of the reaction solution is adjusted to 8 by addition of sodium carbonate (0.29 kg dissolved in water (2.9 L)) and stirred for about 60 minutes for solid separation. The separated solid is filtered, washed with water (2.9 L), and suction dried to afford the title compound.

### Example 7

### Preparation of {3-[2-methoxy-4-methoxycarbonyl-benzyl]-1-methyl-1H-indol-5-yl}-carbamic acid cyclopentyl ester (Formula XV)

Methyl 4-((5-amino-1-methyl-1H-indol-3-yl)methyl)-3-methoxybenzoate (7 kg), N-methyl morpholine (2.5 kg), and toluene (35 L) are charged and the reaction mass is stirred for about 20 minutes. Cyclopentylchloroformate (3.8 kg) dissolved in toluene (4 L) is charged into the reaction mass at 35° C. The resultant reaction mass is stirred for about 60 minutes until completion of the reaction. The solvent is distilled off completely under vacuum below 65° C. Methanol (7 L) is charged and distilled off under vacuum below 65° C. To the obtained crude, methanol (35 L) is charged. The reaction solution is heated to about 65° C. and stirred for about 40 minutes. The resultant reaction solution is cooled to about 0° C. and stirred for about 50 minutes for solid separation. The obtained solid is filtered and washed with methanol (7 L). The solid is dried under vacuum at 60° C. for about 6 hours to afford the title compound.

HPLC purity: 99.84%

### Example 8

### Preparation of {3-[2-methoxy-4-carboxy-benzyl]-1-methyl-1H-indol-5-yl}-carbamic acid cyclopentyl ester (Formula II)

{3-[2-methoxy-4-methoxycarbonyl-benzyl]-1-methyl-1H-indol-5-yl}-carbamic acid cyclopentyl ester (8 kg), methanol (48 L), water (12 L), and lithium hydroxide monohydrate (1.2 kg) are charged and the reaction mass is stirred for about 20 minutes. The resultant reaction is heated to about 70° C. and stirred for about 3 hours until completion of the reaction. After completion of the reaction, the reaction mass is cooled to about 25° C. and the pH of the reaction mass is adjusted to about 1 to about 2 by addition of hydrochloric acid (4 L dissolved in 7 L of water) at about 25° C. The obtained reaction mass is stirred for about 50 minutes. The obtained solid is filtered and washed with water (16 L). The obtained solid is charged into a clean reactor containing methanol (32 L), cooled to about 0° C., and stirred for about 30 minutes. The resultant solid is filtered, washed with methanol (16 L), and suction dried for about 30 minutes. The solid is dried at about 65° C. for 6 hours to afford the title compound.

HPLC purity: 99.20%

### Example 9

### Preparation Zafirlukast Acetonitrile Solvate (Formula IA)

{3-[2-methoxy-4-carboxy-benzyl]-1-methyl-1H-indol-5-yl}-carbamic acid cyclopentyl ester (7 kg), dimethyl amino pyridine (2.5 kg), dicyclohexyl carbodiimide (3.9 kg), and dichloromethane (70 L) are charged and stirred for about 15 minutes. O-toluene sulphonamide (3.4 kg) is added at 30° C. and the reaction solution is stirred for about 4 hours at 30° C. The unwanted separated solid is filtered and the reaction solution is washed with dichloromethane (2×12 L). The obtained organic layer is washed with dilute hydrochloric acid and water and separated. The obtained organic solution is distilled off completely under vacuum below 45° C. Acetonitrile (14 L) is added and distilled off completely under vacuum. The obtained crude is cooled to 30° C., acetonitrile (35 L) is added and heated to 85° C., and stirred for about 40 minutes. The resultant reaction solution is cooled to 35° C. and stirred for about 55 minutes. The separated solid is filtered, washed with acetonitrile (7 mL), and suction dried for about 30 minutes. The obtained wet compound is again charged into a reactor containing methanol (84 L), heated to about 60° C., and stirred for about 30 minutes. The reaction solution is cooled to 30° C. and stirred for about 50 minutes for solid separation. The obtained solid is filtered, washed with methanol (7 L), and suction dried for 30 minutes. The solid is dried under vacuum at 70° C. for about 4 hours to afford the title compound.

HPLC Purity: 97.5%

### Example 10

### Purification of Zafirlukast Acetonitrile Solvate

Zafirlukast acetonitrile solvate (7 kg) and dichloromethane (112 L) are stirred about 30 min at 25° C. for complete dissolution. Silica gel (14 kg) is added into the reaction solution, stirred for about 60 minutes at 30° C., and filtered. The silica gel bed is washed with dichloromethane (2×70 L). The obtained solvent is distilled off completely under vacuum below 45° C. The obtained crude is cooled to 25° C. and acetonitrile (42 L) is added and distilled off completely under vacuum below 80° C. The obtained crude is cooled to 25° C. and acetonitrile (84 L) is added. The reaction solution is heated to 80° C. and stirred for about 40 minutes. The resultant reaction solution is cooled to 30° C. and stirred for about 50 minutes for solid separation. The separated solid is filtered, washed with acetonitrile (21 L), and suction dried for about 30 minutes. The obtained solid is dried at 75° C. under vacuum for about 4 hours. The obtained dry solid is added to a reactor contain dichloromethane (67 L) and stirred for about 15 minutes at 30° C. for complete dissolution. Silica gel (8.4 kg) is charged into the reaction solution and stirred for about 60 minutes at 30° C. The silica gel bed is filtered and washed with dichloromethane (2×35 L). The obtained solvent is distilled off completely under vacuum below 45° C. The obtained crude is cooled to 25° C. and acetonitrile (8 L) is added and distilled off completely under vacuum below 80° C. The obtained crude is cooled to 25° C. and acetonitrile (22 L) is added. The reaction solution is heated to 80° C. and stirred for about 40 minutes. The resultant reaction solution is cooled to 30° C. and stirred for about 50 minutes for solid separation. The separated solid is filtered, washed with acetonitrile (4 L), and suction dried for about 30 minutes. The obtained solid is dried at 75° C. under vacuum for about 4 hours.

HPLC Purity: 99.69%

### Example 11

### Preparation of Amorphous Zafirlukast by Spray Drying

Zafirlukast (2 kg) and methylene chloride (30 L) are charged and stirred for about 15 minutes at 30° C. for complete dissolution. The resultant clear solution is passed through a microfilter to make it particle free. The microfilter is washed with dichloromethane (4 L). The obtained reaction solutions are fed into a spray dryer having an inlet nitrogen temp of 65° C. and a pressure less than 3 kg. The obtained powder is dried under vacuum for about 10 hours at 70° C.

HPLC purity: 99.80%

### Example 12

### Preparation of Amorphous Zafirlukast by Rotary Evaporation Using Dichloromethane as the Solvent

Zafirlukast (90 g) and dichloromethane (1350 mL) are heated to about 40° C. and stirred for about 30 minutes for complete dissolution. The resultant reaction solution is filtered through cloth and paper and washed with dichloromethane (180 mL). The obtained clear solution is distilled off completely under vacuum at 45° C. for about 2-3 hours. The resultant solid is micronized and dried under vacuum at about 55° C. to afford 78 g of amorphous zafirlukast.

HPLC purity: 99.77%

### Example 13

### Preparation of Amorphous Zafirlukast by Rotary Evaporation Using Acetone as the Solvent

Zafirlukast (10 g) and acetone (150 mL) are heated to about 55° C. and stirred for about 30 minutes for complete dissolution. The resultant reaction solution is filtered through cloth and paper and washed with dichloromethane (50 mL). The obtained clear solution is distilled off completely under vacuum at 75° C. for about 2-3 hours. The obtained solid is dried under vacuum at about 75° C. to afford 9.2 g of amorphous zafirlukast.

HPLC purity: 99.60%

### Example 14

### Stability Studies of Amorphous Zafirlukast

A sample of amorphous zafirlukast prepared using the above process was subjected to different conditions to determine the stability of the mixture under stress conditions.

The sample was packed in a clear polyethylene bag, tied with a tag, placed in a black polyethylene bag along with a silica pouch, which was filled with nitrogen, then sealed in a triple laminated bag, and finally sealed and stored in an HDPE drum.

The results are tabulated is Table 2 below:

TABLE 2

| Storage conditions | Amorphous form |
|---|---|
| Long-term storage for 3 months. | No change in XRD spectra |
| Storage at 25 + 5 C. and 60 + 5% RH | No change in XRD spectra |
| Storage at 40 + 5 C. and 70 + 5% RH | No change in XRD spectra |

The consistency in the XRD pattern after subjecting the sample to long term storage conditions shows that there is no interconversion of forms during long term storage.

### Example 15

### Preparation of Crystalline Zafirlukast Form X Using Ethyl Acetate as the Solvent

Zafirlukast (25 g) and ethyl acetate (75 mL) are charged and heated to about 70° C. for about 45 minutes. The resultant reaction solution is cooled to 25° C. and stirred for about 45 minutes for solid separation. The obtained solid is filtered, washed with ethyl acetate (75 mL), and suction dried for 10 minutes. The solid is dried under vacuum at 55° C. for about 3 hours to afford 22 g of crystalline zafirlukast Form X.

HPCL purity: 99.79%

What is claimed is:

1. A process for the formulation of a pharmaceutical composition comprising zafirlukast, the process comprising the steps of:
brominating methyl 3-methoxy-4-methylbenzoate of Formula IX using a mixture of hydrogen bromide and a peroxide to afford methyl 3-methyloxy-4-bromomethylbenzoate of Formula VIII; and
condensing the methyl 3-methyloxy-4-bromomethylbenzoate of Formula VIII with 1-methyl-5-nitro-1H-indole of Formula VII in the presence of a suitable catalyst to afford methyl 3-methoxy-4-(1-methyl-5-nitro-1H-indol-3-yl)methylbenzoate of Formula VI

2. The process of claim 1, wherein the peroxide is hydrogen peroxide.

3. The process of claim 1, wherein the catalyst comprises copper oxide, zinc oxide, magnesium oxide, or calcium oxide.

4. The process of claim 1, further comprising the steps of:
providing methyl 3-methoxy-4-(1-methyl-5-amino-1H-indol-3-yl)methylbenzoate of Formula V; and
purifying the methyl 3-methoxy-4-(1-methyl-5-amino-1H-indol-3-yl)methylbenzoate by recrystallization thereof from an organic solvent.

5. The process of claim 4, wherein the organic solvent is ethyl acetate.

6. The process of claim 4, wherein the purified methyl 3-methoxy-4-(1-methyl-5-amino-1H-indol-3-yl)methylbenzoate has a purity of at least 99% by HPLC.

7. The process of claim 1, further comprising the steps of:
providing a crude zafirlukast; and
treating the crude zafirlukast with an adsorbent to afford zafirlukast substantially free of process related impurities.

8. The process of claim 7, wherein the treated zafirlukast has a purity of at least 99% by HPLC.

9. The process of claim 1, further comprising the steps of:
providing a solution of zafirlukast in a volatile solvent; and
removing the solvent by spray drier or rotary evaporation to afford amorphous zafirlukast.

10. The process of claim 1, further comprising the step of providing zafirlukast having a mean particle size of less than or equal to about 300 μm.

11. A compound selected from the group consisting of:

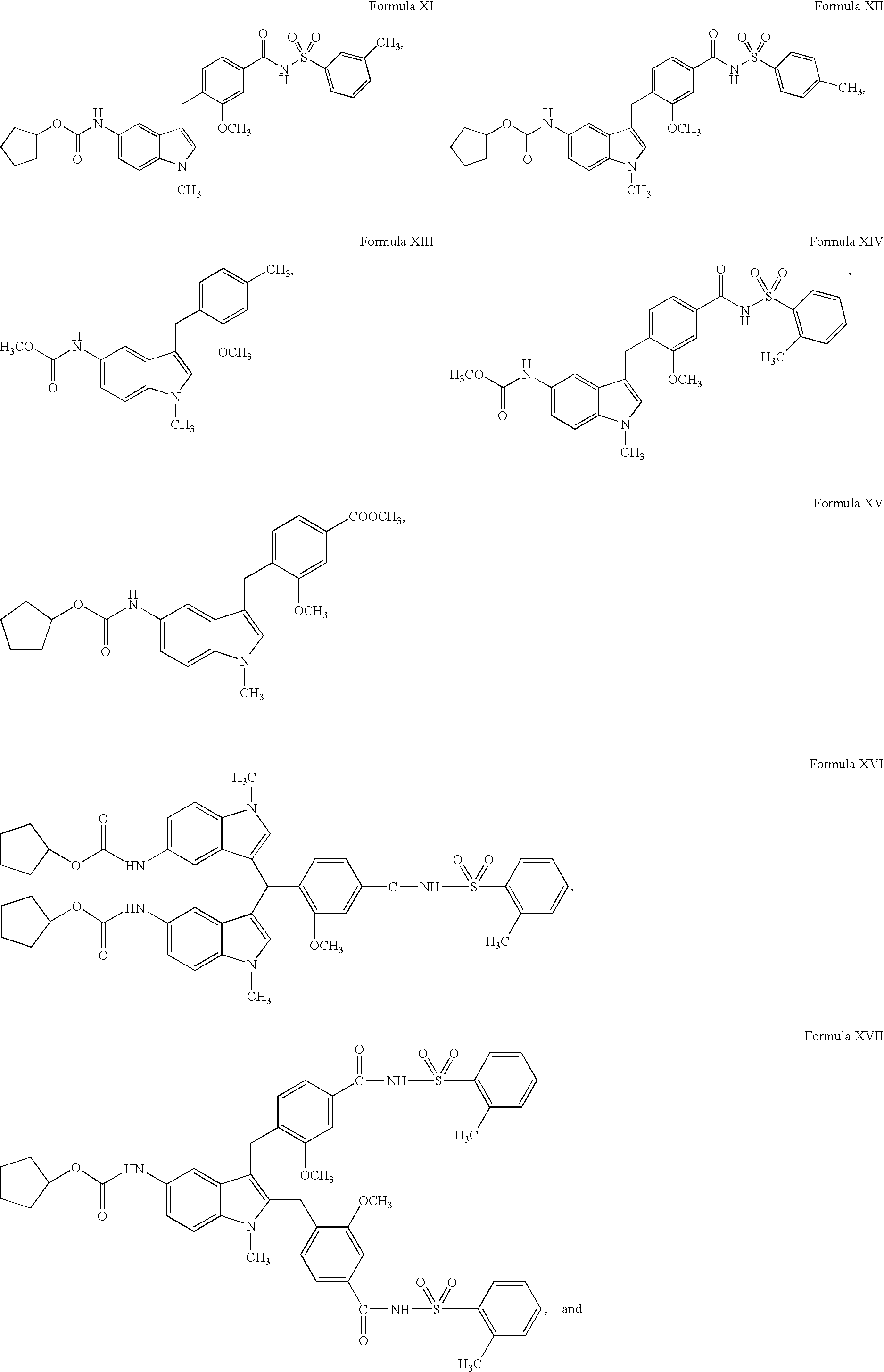

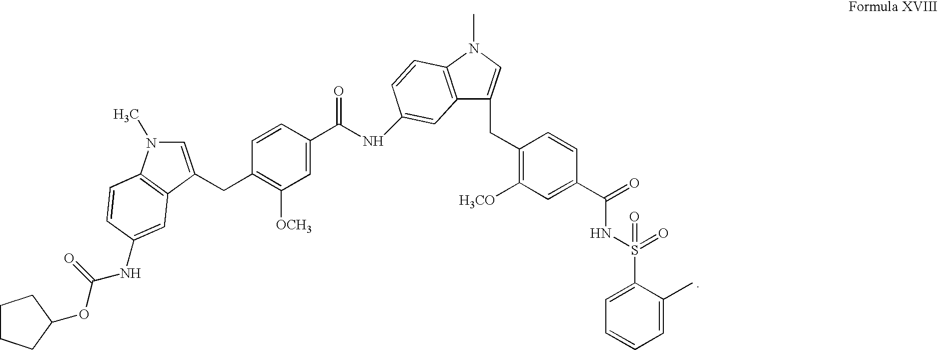

12. A method for analyzing zafirlukast or its related intermediates comprising the step of providing a reference standard compound, the reference standard compound comprising: 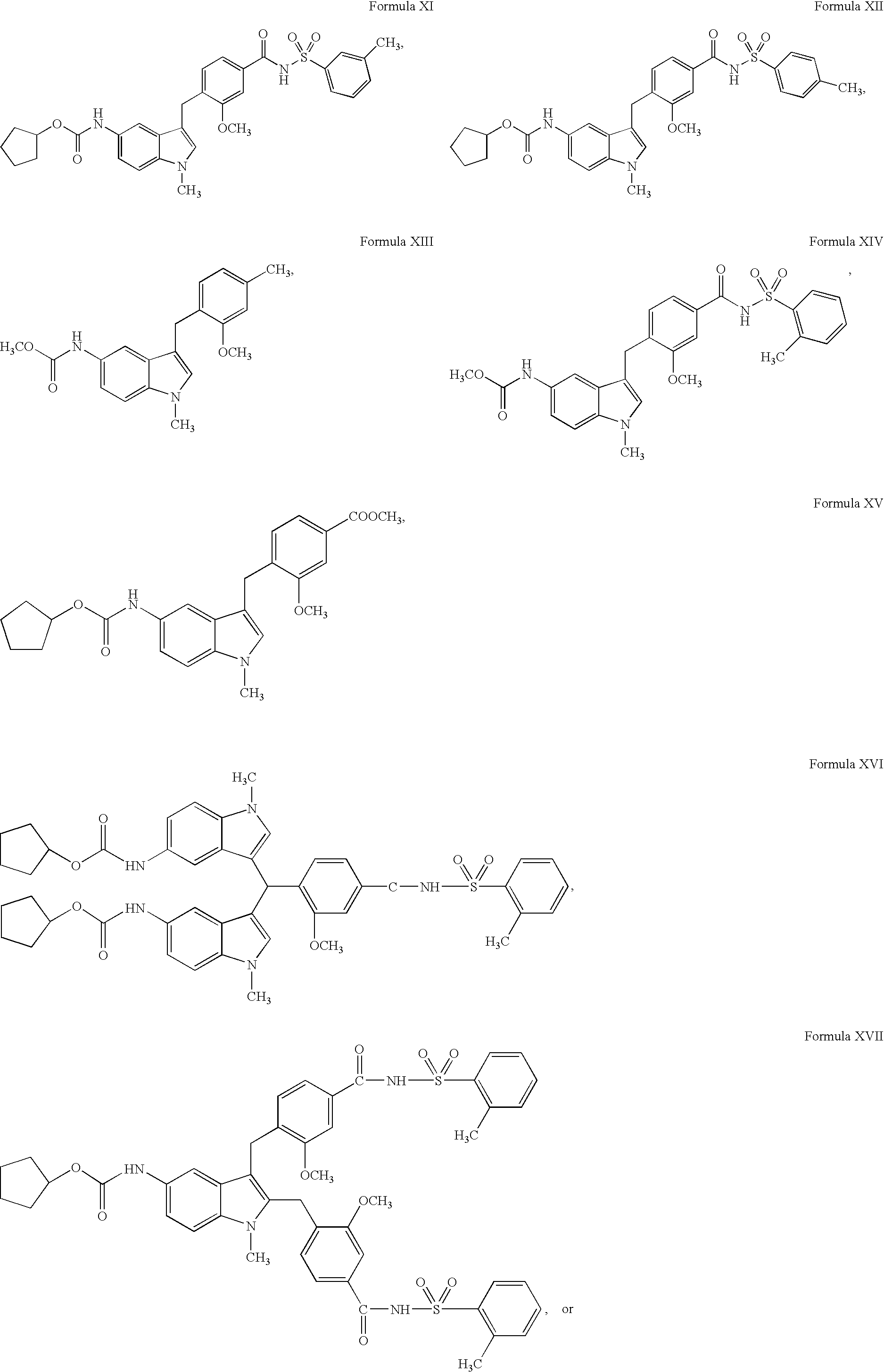 [chemical structure]
